(19) United States
(12) Patent Application Publication
(10) Pub. No.: US 2025/0340949 A1
(43) Pub. Date: Nov. 6, 2025

(54) PCR AMPLIFICATION OF PS-MODIFIED DNA FOR CHEMICAL PROBE READOUT

(71) Applicant: Verily Life Sciences LLC, Dallas, TX (US)

(72) Inventors: Manali Dwarakanath, Fremont, CA (US); Benjamin Krasnow, Redwood City, CA (US); Jasper Lodewyk Steyn, San Bruno, CA (US)

(21) Appl. No.: 19/194,660

(22) Filed: Apr. 30, 2025

Related U.S. Application Data
(60) Provisional application No. 63/641,609, filed on May 2, 2024.

(51) Int. Cl. C12Q 1/6886

(52) U.S. Cl. CPC C12Q 1/6886; C12Q 2600/106; C12Q 2600/156

(57) ABSTRACT

Provided herein are methods for detecting the presence or progression of a disease in a subject as well as a method for determining the efficacy of a therapy for the disease by administering to the subject a probe comprising a phosphorothioated DNA barcode sequence, wherein the DNA barcode sequence comprises about 36 nucleotides to about 72 nucleotides, and wherein the DNA barcode is released when the disease is present in the subject; obtaining a biological sample containing the released DNA barcode from the subject; and amplifying and detecting the DNA barcode in the biological sample.

### CROSS REFERENCE TO RELATED APPLICATIONS

The present application claims the benefit of U.S. Provisional Application No. 63/641,609 filed on May 2, 2024, the entire disclosure of which is hereby incorporated herein by reference in its entirety for all purposes.

### REFERENCE TO A SEQUENCE LISTING SUBMITTED AS XML VIA PATENT CENTER

The instant application contains a Sequence Listing that has been filed electronically in. xml format and is hereby incorporated by reference in its entirety. Said.xml copy, created on May 2, 2024, is named Ver-496PV1-1422117_seqlist.xml and is 5,000 bytes in size.

### BACKGROUND

Chemical probes, in the context of healthcare, are substances that, when administered to a subject, test for the state of a disease or physiology in the subject. Simple chemical probes include a specific sugar solution that, when consumed by a subject, results in detectable hydrogen in the subject's exhaled breath if the subject has certain bacterial infections. More complex chemical probes have been developed, such as probes that include a DNA barcode released into a bodily fluid upon enzymatic cleavage by an enzyme associated with the disease. However, the amount of barcode released is often in miniscule amounts that require extremely high sensitivity assays to detect the barcode. In addition, DNA barcodes are quickly degraded. Although DNA barcodes can be modified to comprise phosphorothioated bonds in order to increase the half-life of the barcode, the addition of such a modification makes the DNA barcode difficult to detect by PCR amplification. To date, these limitations have proven problematic for detecting disease conditions such as cancer using chemical probes.

### SUMMARY

Provided herein is a method for detecting the presence of a disease, such as cancer, in a subject by detecting a DNA barcode released from a probe in the presence of the disease. The method includes administering to the subject a probe comprising a phosphorothioated DNA barcode sequence, wherein the DNA barcode sequence comprises about 36 nucleotides to about 72 nucleotides and wherein the DNA barcode is released when the disease is present in the subject. A biological sample (e.g., a blood or urine sample) containing the released DNA barcode is then obtained from the subject, and the DNA barcode sequence in the biological sample is amplified and detected. Detection of the DNA barcode sequence in the biological sample indicates the presence of the disease in the subject. Optionally, detecting an amount (or level) of the DNA barcode sequence indicates the level of disease or the effect of treatment.

Also provided herein is a method of treating the subject with a disease, such as cancer. The method comprises utilizing the method for detecting the presence of the disease in the subject by administering a probe with a phosphorothioated DNA barcode sequence to the subject and, if the disease is determined to be present, administering a treatment (e.g., a therapeutic agent, radiation, stem cell transplant, etc.) to the subject.

Optionally, the method for detecting the presence of the disease in the subject by administering a probe with a phosphorothioated DNA barcode sequence to the subject may be performed at multiple time points to detect changes in the level of the DNA barcode sequence or before and after one or more treatments. An increase in the level of the released DNA barcode over time or after treatment(s) indicates progression of the disease or ineffective treatment, whereas a decrease in the level of the released DNA barcode over time or after treatment(s) indicates reduction or remission of the disease or efficacy of one or more treatments given to the subject.

Also provided is a kit comprising a probe with a phosphorothioated DNA barcode of about 36 nucleotides to about 72 nucleotides. The kit can further include a forward primer comprising about 18 nucleotides that hybridize to about 18 nucleotides at the 3′ end of the DNA barcode and a reverse primer comprising about 18 nucleotides that hybridize to about 18 nucleotides at the 5′ end of the DNA barcode.

The details of one or more embodiments are set forth in the description below. Other features, objects, and advantages will be apparent from the description and from the claims.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows the average Ct value plotted for various phosphorothioate (PS)-modified DNA barcodes (SEQ ID NO: 1-3) at various input concentrations. The 62 nucleotide partially modified versions (e.g., 5′3′ modified (5′3′_mod), every 2^{nd} nucleotide (2^{nd}), every 4^{th} nucleotide (4^{th}), and fully modified except priming regions (noprim) have similar Ct values to the unmodified 72 nucleotide (no-mod), 62 nucleotide (no_mod), and 46 nucleotide (no_mod) barcodes. Further, the 72 nucleotide and 62 nucleotide fully PS-modified DNA (all) have significantly higher Ct values compared to their corresponding non-modified and partially modified barcodes. Unexpectedly, this trend is not the same for the 46 nucleotide fully modified barcode (all), which has a similar Ct value compared to its corresponding non-modified barcodes.

FIG. 2 shows the average Ct value plotted for PS-modified DNA barcodes (SEQ ID NO: 1-4) at an input concentration of 10 pM compared to a negative control sample. The Ct values for both the 62 nucleotide and the 72 nucleotide fully modified barcodes (all) are higher compared to their corresponding barcodes without modifications (no_mod). In addition, the Ct trend shows that, as the length of the fully-PS modified barcodes decrease, the average Ct value becomes lower.

FIGS. 3A-3H shows electropherogram graphs from fragment analysis for plotting DNA fragment size (base pairs) by the fluorescence intensity (RFU) signal from the various sized barcodes. FIGS. 3A and 3B show the fragment analysis for the 36 nucleotide barcodes without any modifications (SEQ ID No. 4; FIG. 3A) and fully modified (SEQ ID No. 4; FIG. 3B). The prominent peak at 137 and 135, respectively, correlate with the expected amplicon length of 130 base pairs (including the 18-nucleotide barcode specific region and the NGS flanking sequences). Similarly, the fragment analysis for the 46 nucleotide barcodes without any modifications (SEQ ID No. 3; FIG. 3C) and fully modified (SEQ ID No. 3; FIG. 3D) also have their prominent peaks correlated with the expected amplicon length of 140 base pairs when the 18-nucleotide barcode specific region and the NGS flanking sequences are also included. Both the 62 nucleotide barcode without modifications (SEQ ID No. 2; FIG. 3E) and the 62 nucleotide barcode that is fully modified (SEQ ID No. 2; FIG. 3F) have their prominent peak correlate with the expected amplicon length of 156 base pairs, as do both the 72 nucleotide barcodes without (SEQ ID No. 1; FIG. 3G) and with (SEQ ID No. 1; FIG. 3H) modifications.

### DETAILED DESCRIPTION

The following description recites various aspects and embodiments of the present compositions and methods. No particular embodiment is intended to define the scope of the methods. Rather, the embodiments merely provide non-limiting examples that are at least included within the scope of the disclosed methods. The description is to be read from the perspective of one of ordinary skill in the art; therefore, information well known to the skilled artisan is not necessarily included.

Detection of DNA barcodes in a blood sample (including whole blood, plasma, or serum) of a subject is notoriously challenging given the rapid degradation of DNA and the miniscule amounts of the DNA barcode present. To improve DNA barcode stability, phosphorothioate bonds are added to the DNA barcode; however, their addition makes it far more difficult to detect the DNA barcodes via amplification methods due to weak chemical association of the polymerase to the phosphorothioated DNA barcode. To this end, a specific range of phosphorothioated DNA barcode lengths are described that can be amplified via PCR from a biological sample collected from a subject.

Thus, provided herein is a method for detecting the presence of a disease by detecting the presence of or determining the level of a released DNA barcode sequence in a biological sample form a subject. The method includes administering a probe comprising a phosphorothioated DNA barcode sequence to the subject. The DNA barcode sequence comprises about 36 nucleotides to about 72 nucleotides in length and can further comprise (1) a priming sequence at the 5′ end of the DNA barcode sequence and a priming sequence at the 3′ end of the DNA barcode sequence, (2) a unique molecular identifier (UMI) sequence, or (3) any combination thereof. The DNA barcode is incorporated into a probe that is administered to a subject. The DNA barcode sequence, thus, can include a priming sequence comprising about 18nucleotides at the 5′ end of the DNA barcode sequence, a priming sequence comprising about 18nucleotides at the 3′ end of the DNA barcode sequence, and a unique molecule identifier. The DNA barcode sequence may be either single stranded or double stranded.

The present phosphorothioated DNA barcode and the probe containing it overcome amplification difficulties usually presented by phosphorothioated DNA. Described herein is a specific range of phosphorothioated DNA barcode lengths in which amplification occur. The DNA barcode may comprise about 36 nucleotides to about 72 nucleotides in length. Optionally, the DNA barcode sequence may comprise about 36 nucleotides to about 62 nucleotides in length, or about 36 nucleotides to about 46 nucleotides in length. The lower limit on nucleic acid length may be established by the length of the priming sequence or priming regions of the DNA barcode sequence. For example, the minimum priming sequence at both the 3′ and 5′ ends of the DNA barcode sequence may be 18 nucleotides, resulting in a minimum DNA barcode length of 36 nucleotides. The upper limit on nucleic acid length may be measured by how effective the amplification method performs. The effectiveness of amplification is defined by how robust the amplification is from a test DNA probe as compared to a control probe (e.g., of greater length or having more phosphorothioated bonds). For example, a DNA barcode sequence level or amount may be quantified by RT-qPCR and shown to have significantly higher cycle threshold (Ct) values compared to one or more different DNA barcode sequences. In this context, the DNA barcode sequence with the higher Ct values is considered less effective. One of skill in the art will appreciate that effective amplification will vary in various experimental conditions.

The phosphorothioated DNA barcode sequence comprises at least one phosphorothioated modification. Phosphorothioates (or S-oligos) are a variant of normal DNA in which one of the nonbridging oxygens is replaced by sulfur. More specifically, the phosphorothioate bond substitutes a sulfur atom for a non-bridging oxygen in the phosphate backbone of an oligonucleotide, rendering the nucleotide linkage resistant to nuclease degradation. Phosphorothioates can be introduced at either or both the 5′-and/or 3′-end of the oligo to inhibit exonuclease degradation. The synthesis of phosphorothioate containing oligonucleotides is described, for example, in Verma and Eckstein (1998), Modified Oligonucleotides: Synthesis and Strategy for Users, Annu. Rev. Biochem. 67:99-134, and in Nawrot and Rebowska (2009) DNA Oligonucleotides Containing Stereodefined Phosphorothioate Linkages in Selected Positions, Curr. Protoc. Nucleic Acid Chem., Chapter Unit 4.34. These references are incorporated herein in their entireties for the phosphorothioate containing oligonucleotides and synthesis methods disclosed therein.

In the methods provided herein, the DNA barcode sequences contain one or more phosphorothioate-modified bases (e.g., phosphorothioated modifications). The number of phosphodiester linkages replaced by phosphorothioates in any given DNA barcode sequence can range from one to all of the phosphodiester bonds being replaced by phosphorothioates. By way of example, the DNA barcode sequence may comprise a phosphorothioated modification at the 3′ end of the DNA barcode sequence and a phosphorothioated modification at the 5′ end of the DNA barcode sequence. Optionally, the DNA barcode sequence may comprise a phosphorothioated modification at various intervals, for example, at every nucleotide, at every second nucleotide, at every fourth nucleotide, at every fifth nucleotide, at every sixth nucleotide, at every seventh nucleotide, at every either eighth nucleotide, at every ninth nucleotide, at every tenth nucleotide, and so on. A further example may include that the priming sequences of the DNA barcode sequence are not phosphorothioated modified. Examples of DNA barcode sequence length in combination with the various phosphorothioated modification patterns are shown in Table 1.

TABLE 1
Phosphorothioate Modified DNA Barcode Designs

| | | | SEQ |
|---|---|---|---|
| DNA Barcode | | | ID |
| description | Probe Sequence | Code | NO. |
| Original full- | AGCGTCAGATGTGTATAAGAGACAGNN | 72 nt_no_mod | 1 |
| length (72 nt) | NNNNNNNNCTCTCTATTAAGGCGAAGATC | | |
| | GGAAGAGCGGTTCAGC | | |
| 5′ and 3′ PS | A*G*C*GTCAGATGTGTATAAGAGACAG | 72 nt_5′3′_mod | 1 |
| | NNNNNNNNNNCTCTCTATTAAGGCGAAG | | |
| | ATCGGAAGAGCGGTTC*A*G*C | | |
| Every 2^{nd} base | AG*CG*TC*AG*AT*GT*GT*AT*AA*GA* | 72 nt_2nd | 1 |
| modified | GA*CA*GN*NN*NN*NN*NN*NC*TC*TC* | | |
| | TA*TT*AA*GG*CG*AA*GA*TC*GG*AA*G | | |
| | A*GC*GG*TT*CA*GC | | |
| Every 4^{th} base | AGCG*TCAG*ATGT*GTAT*AAGA*GACA | 72 nt_4th | 1 |
| modified | *GNNN*NNNN*NNNC*TCTC*TATT*AAGG | | |
| | *CGAA*GATC*GGAA*GAGC*GGTT*CAG | | |
| | C | | |
| Modifications | AGCGTCAGATGTGTATA*A*G*A*G*A*C | 72 nt_noprim | 1 |
| outside of priming | *A*G*N*N*N*N*N*N*N*N*N*N*C*T*C*T | | |
| region | *C*T*A*T*T*A*A*G*G*C*G*A*A*G*A*T* | | |
| | CGGAAGAGCGGTTCAGC | | |
| All bases modified | A*G*C*G*T*C*A*G*A*T*G*T*G*T*A*T* | 72 nt_all | 1 |
| | A*A*G*A*G*A*C*A*G*N*N*N*N*N*N*N | | |
| | *N*N*N*C*T*C*T*C*T*A*T*T*A*A*G*G* | | |
| | C*G*A*A*G*A*T*C*G*G*A*A*G*A*G*C* | | |
| | G*G*T*T*C*A*G*C | | |
| Original full- | AGCGTCAGATGTGTATAAGAGACAGCTC | 62 nt_no_mod | 2 |
| length (62 nt) | TCTATTAAGGCGAAGATCGGAAGAGCGG | | |
| | TTCAGC | | |
| 5′ and 3′ PS | A*G*C*GTCAGATGTGTATAAGAGACAG | 62 nt_5′3′_mod | 2 |
| | CTCTCTATTAAGGCGAAGATCGGAAGAGC | | |
| | GGTTC*A*G*C | | |
| Every 2^{nd} base | AG*CG*TC*AG*AT*GT*GT*AT*AA*GA* | 62 nt_2nd | 2 |
| modified | GA*CA*GC*TC*TC*TA*TT*AA*GG*CG*AA | | |
| | *GA*TC*GG*AA*GA*GC*GG*TT*CA*GC | | |
| Every 4^{th} base | AGCG*TCAG*ATGT*GTAT*AAGA*GACA | 62 nt_4th | 2 |
| modified | *GCTC*TCTA*TTAA*GGCG*AAGA*TCGG* | | |
| | AAGA*GCGG*TTCA*GC | | |
| Modifications | AGCGTCAGATGTGTATA*A*G*A*G*A*C | 62 nt_noprim | 2 |
| outside of priming | *A*G*C*T*C*T*C*T*A*T*T*A*A*G*G*C* | | |
| region | G*A*A*G*A*T*C*GGAAGAGCGGTTCAG | | |
| | C | | |
| All bases modified | A*G*C*G*T*C*A*G*A*T*G*T*G*T*A*T* | 62 nt_all | 2 |
| | A*A*G*A*G*A*C*A*G*C*T*C*T*C*T*A*T | | |
| | *T*A*A*G*G*C*G*A*A*G*A*T*C*G*G*A* | | |
| | A*G*A*G*C*G*G*T*T*C*A*G*C | | |
| Original full- | AGCGTCAGATGTGTATAAGAGACAGAG | 46 nt_no_mod | 3 |
| length (46 nt) | ATCGGAAGAGCGGTTCAGC | | |
| 5′ and 3′ PS | A*G*C*GTCAGATGTGTATAAGAGACAG | 46 nt_5′3′_mod | 3 |
| | AGATCGGAAGAGCGGTTC*A*G*C | | |
| Every 2^{nd} base | AG*CG*TC*AG*AT*GT*GT*AT*AA*GA* | 42 nt_2nd | 3 |
| modified | GA*CA*GA*GA*TC*GG*AA*GA*GC*GG* | | |
| | TT*CA*GC | | |
| Every 4^{th} base | AGCG*TCAG*ATGT*GTAT*AAGA*GACA | 46 nt_4th | 3 |
| modified | *GAGA*TCGG*AAGA*GCGG*TTCA*GC | | |
| Modifications | AGCGTCAGATGTGTATA*A*G*A*G*A*C | 46 nt_noprim | 3 |
| outside of priming | *A*G*A*G*A*T*C*GGAAGAGCGGTTCA | | |
| region | GC | | |
| All bases modified | A*G*C*G*T*C*A*G*A*T*G*T*G*T*A*T* | 46 nt_all | 3 |
| | A*A*G*A*G*A*C*A*G*A*G*A*T*C*G*G* | | |
| | A*A*G*A*G*C*G*G*T*T*C*A*G*C | | |
| Original full- | AGCGTCAGATGTGTATAATCGGAAGAGC | 36 nt_no_mod | 4 |
| length (36 nt) | GGTTCAGC | | |
| All bases modified | A*G*C*G*T*C*A*G*A*T*G*T*G*T*A*T* | 36 nt_all | 4 |
| | A*A*T*C*G*G*A*A*G*A*G*C*G*G*T*T* | | |
| | C*A*G*C | | |

Table notations:
Underlined nucleotides indicate the 5′ and 3′ priming sequences.
Normal text nucleotides indicate a variable sequence outside the priming regions.
Bolded nucleotides indicate the UMI sequence.
Italicized nucleotides indicate sequences used for next generation sequencing (NGS) and/or library management (e.g., Sseq + Nseq barcode).
Phosphorothioate Modifications denoted by *

The presence and/or amount of the released DNA barcode sequence can serve as an indicator for disease diagnosis, progression, and/or treatment response. To identify the presence and/or to measure the level of the released DNA barcode sequence, a biological sample containing the released DNA barcode is collected from a subject. The biological sample derived from a subject includes, but is not limited to, any cell, tissue, or biological fluid. The sample can be, but is not limited to, whole blood, plasma, serum, sputum, urine, saliva, bronchoalveolar lavage fluids, biopsy (e.g. tissue or cells isolated from organ tissue, for example, from lung, liver kidney, skin etc.), vaginal secretion, nasal secretion, skin, gastric secretion, or bone marrow specimens. Optionally, the biological sample is a blood sample (whole blood, plasma, or serum) or a urine sample collected from the subject.

In the methods provided herein, the DNA barcode sequence can be detected by amplification of the DNA barcode in the biological sample (e.g., urine) collected from the subject. Amplification may be facilitated by a priming sequence at the 5′ end of the DNA barcode sequence and a priming sequence at the 3′ end of the DNA barcode sequence. The priming sequences at both the 5′ end and the 3′ end of the DNA barcode sequence may comprise about 18 nucleotides. The process for amplification of the DNA barcode sequence can be achieved by any means known in the art, for example, via polymerase chain reaction (PCR). Exemplary PCR techniques include, but are not limited to, endpoint PCR, real-time PCR (quantitative PCR or qPCR), reverse-transcriptase PCR (RT-PCR), multiplex PCR, nested PCR, and high-fidelity PCR. See, or example, Eftekhari et al. “A Comprehensive Review of Detection Methods for SARS-CoV-2,” Microorganisms 2021, 9, 232. The primers and probes for amplification can be purchased or prepared by any means known in the art, including automated processes. In some embodiments, the primers and probes are designed for specificity for the DNA barcode sequence, as disclosed herein.

The DNA barcode sequence may be amplified by performing PCR using a forward primer comprising a nucleic acid sequence that comprises about 18 nucleotides hybridizes to the priming sequence at the 3′ end of the DNA barcode and a reverse primer comprising a nucleic acid sequence that comprises about 18 nucleotides hybridizes to the priming sequence at the 5′ end of the DNA barcode. The term primer, either forward or reverse primers, refers to an oligonucleotide, whether occurring naturally as in a purified restriction digest or produced synthetically, that is capable of acting as a point of initiation of synthesis when placed under conditions in which synthesis of a primer extension product complementary to a nucleic acid strand is induced, (i.e., in the presence of nucleotides and an inducing agent such as DNA polymerase and at a suitable temperature and pH). The primer is preferably a single stranded oligonucleotide for maximum efficiency in amplification. The primer should be sufficiently long to prime the synthesis of extension products in the presence of the inducing agent. The exact lengths of the primers will depend on many factors, including temperature, source of primer and the use of the method. In one exemplary embodiment, the DNA barcode sequence may be amplified using a pair of forward and reverse primers, wherein the forward primer comprises a nucleic acid sequence comprising about 18 nucleotides that hybridize to about 18 nucleotides at the 3′ end of the DNA barcode and the reverse primer comprises a nucleic acid sequence comprising about 18 nucleotides that hybridize to about 18 nucleotides at the 5′ end of DNA barcode.

The UMI sequence in the DNA barcode is a unique nucleotide sequence that is used to distinguish one DNA barcode from another. The UMI may be any number of nucleotides of sufficient length to distinguish the UMI from other UMI. For example, a UMI may be from about 5 to about 10 random nucleotides in length, such as 5, 6, 7, 8, 9, or 10 nucleotides in length. UMIs may also be used to count the occurrences of the DNA barcode sequences for absolute molecular counting.

Detection of the amplified DNA barcode sequence in the biological sample (e.g., blood or urine) indicates the presence of a disease or condition in the subject. As used throughout, a disease is an abnormal condition that adversely affects the structure or function of all, substantially all, or part of an organism and is not immediately due to any external injury. In some cases, the presence of a condition of unknown etiology is identified. A disease may be a proliferative disease (e.g., cancer), an infectious disease, a deficiency disease, a hereditary disease, an immune or autoimmune disorder. Infectious diseases include, without limitation, infection by viruses, bacteria, fungi, protozoa, multicellular organisms, and aberrant proteins known as prions. Examples of diseases, without limitation, include cancer, neurodegenerative diseases, autoimmune diseases or disorders; and the like.

Cancer refers to an abnormal state or condition characterized by rapidly proliferating cells. Rapidly proliferating cells may be categorized as pathologic (i.e., characterizing or constituting a disease state) or may be categorized as non-pathologic (i.e., a deviation from normal but not associated with a disease state). Cancer can be characterized by one or more solid tumor or by a liquid tumor (e.g., blood or bone marrow). Cancer also refers to primary or metastatic tumors. Examples of cancer include malignancies of various organ systems, such as lung cancers, breast cancers, thyroid cancers, lymphoid cancers, gastrointestinal cancers, and genito-urinary tract cancers. Cancer can also refer to adenocarcinomas, which include malignancies such as colon cancers, renal-cell carcinoma, prostate cancer and/or testicular tumors, non-small cell carcinoma of the lung, cancer of the small intestine, and cancer of the esophagus. Carcinomas of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An adenocarcinoma refers to a carcinoma derived from glandular tissue or in which the tumor cells form recognizable glandular structures. A sarcoma refers to a malignant tumor of mesenchymal derivation. Melanoma refers to a tumor arising from a melanocyte.

Subject, as used herein, refers to a mammal, such as a human or non-human primate, wherein the mammalian subject can be of any age. In any of the methods set forth herein, the subject can be considered healthy or may be suspected of having a disease, diagnosed with a disease, or receiving treatment for a disease. For example, the subject may be deemed healthy but screened for cancer, may be suspected of having cancer, may be diagnosed with cancer, or may be receiving treatment for cancer.

Administration of the probe comprising the phosphorothioated DNA barcode sequence to the subject may be done using a variety of methods known in the art. For example, the probe may be administered orally, intramuscularly, intravenously (e.g., by intravenous infusion), subcutaneously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, topically, locally, by inhalation, by injection, by infusion, by continuous infusion, by localized perfusion bathing target cells directly, by catheter, by lavage, in creams, or in lipid compositions. The method of administration can vary depending on various factors (e.g., the type, the location, and/or the severity of the condition, disease, or disorder being detected or treated).

Further, the probe may be attached to the surface of or within a nanoparticle for the delivery of the phosphorothioated DNA barcode sequence to the subject. As used throughout, nanoparticles can be, but are not limited to, lipid nanoparticles, for example, liposomes or non- liposomal lipid nanoparticles (for example, lipid nanoparticles with a non-aqueous core (LNPs)), dendrimers, polymeric micelles, nanocapsules or nanospheres, to name a few. Other examples include, but are not limited to, iron oxide nanoparticles, polysaccharide gel nanoparticles and silica nanoparticles.

As used herein, the term liposome refers to an aqueous or aqueous-buffered compartment enclosed by at least one lipid bilayer. Liposomes are capable of carrying aqueous solutions, compounds, drugs or other substances in the compartment (i.e., internal cavity or space, enclosed by at least one lipid bilayer). Liposomes can vary in size, i.e., diameter. For example, a liposome can have a size of about 1000 nanometers (nm) or less. For example, a liposome can have a size of about 50 nm to about 1000 nm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm. Liposomes include liposomes comprising a compartment for encapsulation of an agent, for example, a phosphorothioated DNA barcode sequence. An encapsulated phosphorothioated DNA barcode sequence is a phosphorothioated DNA barcode sequence that is completely or partially located in the interior space of the liposome. For example, in any of the liposomes described herein, at least about 75%, 80%, 85%, 90%, 95% or 99% of the phosphorothioated DNA barcode sequence is incorporated into the interior space of the liposome or into the lipid bilayer of the liposome.

Any of the nanoparticles described herein, for example, liposomes, can contain about 1, 2, 3, 4, 5, 6, 7, 8, 9, 10, 11, 12, 13, 14, 15, 16, 17, 18, 19, 20 mole percent or greater of a phosphorothioated DNA barcode sequence relative to lipid. In other words, the nanoparticles can comprise a 1:100, 2:100, 3:100, 4:100, 5:100, 6:100, 7:100, 8:100, 9:100, 10:100, 11:100, 12:100, 13:100, 14:100, 15:100, 16:100, 17:100, 18:100, 19:100, or 20:100 mole ratio of phosphorothioated DNA barcode sequence to liposomal lipid or greater. As used herein, mole ratio is the ratio between the amounts in moles of two components, for example, the ratio between the number of moles of the targeting molecule and the number of moles of lipid (targeting molecules: moles of lipid) or the number of moles of a phosphorothioated DNA barcode sequence and the number of moles of lipid (moles of phosphorothioated DNA barcode sequence: moles of lipid). And similarly, the nanoparticles can comprise a 0.002:100, 0.05:100, 0.1:100, 0.5:100, 1:100, 2:100, 3:100, 4:100, 5:100, 10:100, 15:100, 20:100, 25:100 mole ratio of targeting protein to liposomal lipid or greater.

Pluralities of two or more of any of the nanoparticles or liposomes described herein are also provided. For example, a plurality of nanoparticles or liposomes can comprise from about two to about 1×10¹⁴ (100 trillion) nanoparticles or liposomes. For example, a plurality can have at least 100, 250, 500, 750, 1000, 5000, 10,000, 25,000, 50,000,100,000, 500,000, 1 million, or more nanoparticles or liposomes. Nanoparticles or liposomes can be made by any suitable method known to or later discovered by one of skill in the art. In general, liposomes can be prepared by a thin film hydration technique followed by a few freeze-thaw cycles. Liposomal suspensions can also be prepared according to methods known to those skilled in the art. Exemplary methods for the preparation of liposomes are described in Akbarzadeh et al. (2013) Liposome: classification, preparation and applications, Nanoscale Res. Lett. 8(1): 102 (2013)) which is hereby incorporated by reference in its entirety for the liposomes and related methods disclosed therein.

In general, a variety of lipid components can be used to make liposomes. These include neutral lipids that exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. Synthetic derivatives of any of the lipids described herein can also be used to make lipid nanoparticles. Lipid nanoparticles can also comprise a sterol, for example, cholesterol. Lipid nanoparticles can also comprise a cationic lipid which carries a net positive charge at about physiological pH. Such cationic lipids include, but are not limited to, N,N-dioleyl-N,N-dimethylammonium chloride (DODAC); N-(2,3-dioleyloxy)propyl-N,N-N-triethylammonium chloride (DOTMA); N,N-distearyl-N,N-dimethylammonium bromide (DDAB); N-(2,3-dioleoyloxy)propyl)-N,N,N-trimethylammonium chloride (DOTAP); 1,2-Dioleyloxy-3-trimethylaminopropane chloride salt (DOTAP.Cl); 3.beta.-(N--(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (DOGS), 1,2-dileoyl-sn-3-phosphoethanolamine (DOPE), 1,2-dioleoyl-3-dimethylammonium propane (DODAP), N,N-dimethyl-2,3-dioleyloxy)propylamine (DODMA), and N-(1,2-dimyristyloxyprop-3-yl)-N,N-dimethyl-N-hydroxyethyl ammonium bromide (DMRIE). Anionic lipids are also suitable for use in lipid nanoparticles described herein. These include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In some examples, the liposome comprises phosphatidylcholine, phosphatidylethanolamine, phosphatidylserine, phosphatidylinositol, phosphatidic acid, phosphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, distearoylphosphatidylcholine (DSPC), dilinoleoylphosphatidylcholine, a 1,2-distearoyl-sn-glycero-3-phosphoethanolamine (DSPE) conjugated polyethylene glycol (DSPE-PEG), a sphingomyelin, cholesterol, or any combination thereof. In some embodiments, PEG can be PEG-molecular weight (MW500) to PEG-MW20000. In addition to being components of the liposomes described herein, any of the lipids described herein can be conjugated to a targeting molecule or a fragment thereof that binds an antigen. In some examples, pegylated versions of any of the lipids described herein can be conjugated to a targeting molecule or a fragment thereof that binds an antigen, for example, a cancer cell antigen.

The probe further comprises an enzymatic cleavage site that links the DNA barcode to the probe. The enzymatic cleavage site is selected as one recognized by an enzyme associated with the disease (e.g., cancer) in the subject. Upon contact of the probe with the enzyme, the DNA barcode is released from the probe. Nonlimiting examples of enzymes specifically over-expressed in a tumor microenvironment include glucuronidase and protease enzymes that target Valine-Alanine and Valine-Cysteine linkers. The released DNA barcode can be present in the blood, urine, or any cellular or extracellular fluid sample, depending on the location of the release and whether the release DNA barcode enters the general circulation and reaches the urinary system, for example. If the probe is delivered in a nanoparticle, the nanoparticle could passively degrade and release the probe. Optionally the nanoparticle could be targeted using a targeting moiety to a certain tissue type such that the probe is selectively released in a particular organ or tissue type. Nanoparticles can be retained by the Enhanced Permeation and Retention (EPR) effect, referring to the progressive accumulation of macromolecular compounds (e.g., nanoparticles above 40 kDa in size) in the tumor vascularized area. Because the nanoparticles accumulate in the vascularized tumor, delivery and retention of nanoparticles containing the probes could be targeted to tumor tissue without the need for targeting moieties. In certain cases, the nanoparticle or probe could be endocytosed into the cell where the DNA barcode is released or exocytosed, reaching for example, the blood and the urinary system over time.

A urine or blood sample containing the released DNA barcode can be processed to extract the phosphorothioate-modified DNA barcode sequence. The DNA barcode sequence may be amplified using any of the above-mentioned amplification methods and detected using DNA detection methods. One example of a DNA detection method includes agarose gel electrophoresis where the DNA is visualized by ethidium bromide or SYBR® green (Thermo Fisher, Waltham, MA). Another example could include a semiquantitative analysis in the event RT-qPCR is used to indirectly detect the DNA.

Also provided herein are methods of treating a disease in a subject. The treatment method includes performing the above method described herein to detect the presence of a disease in the subject. The treatment method optionally includes selecting and administering treatment based on the results of method and/or altering treatment based on the results of the detection method. Treatment refers to improving or slowing onset or progression of one or more symptoms of disease in the subject being treated. Treatment can include providing to the subject an effective amount of a therapeutic agent such as a chemotherapeutic agent, an immunotherapeutic agent, a radiological therapy, and/or cellular therapy (e.g., CAR T cell therapy).

An effective amount of a treatment agent, as used throughout, is defined as any amount necessary to produce a desired physiologic response, for example, reducing or delaying one or more effects or symptoms of a disease or disorder. Effective amounts and schedules for administering the therapeutic agent can be determined empirically, making such determinations within the skill of one in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the species, age, body weight, general health, sex and diet of the subject, the mode and time of administration and severity of the particular condition and can be determined by one of skill in the art. The dosage can be adjusted by the individual physician in the event of any contraindications. Dosages can vary and can be administered in one or more doses.

In some embodiments, the therapeutic agent may be a chemotherapeutic agent or a cocktail of multiple chemotherapeutic agents. In some embodiments the chemotherapeutic agent or cocktail is administered in combination with one or more physical methods (e.g., radiation therapy). The term “chemotherapeutic agents” includes but is not limited to alkylating agents such as thiotepa and cyclosphosphamide, alkyl sulfonates such as busulfan, improsulfan and piposulfan, aziridines such as benzodopa, carboquone, meturedopa, and uredopa, ethylenimines and methylamelamines including altretamine, triethylenemelamine, trietylenephosphoramide, triethylenethiophosphaoramide and trimethylolomelamime, nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas such as carmustine, chlorozotocin, fotemustine, lomustine, nimustine, ranimustine, antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, anti-metabolites such as methotrexate and 5-fluorouracil (5-FU), folic acid analogues such as denopterin, methotrexate, pteropterin, trimetrexate, dideazatetrahydrofolic acid, and folinic acid, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, 5-FU, androgens such as calusterone, dromostanolone propionate, epitiostanol, mepitiostane, testolactone, anti-adrenals such as aminoglutethimide, mitotane, trilostane, folic acid replenisher such as frolinic acid, aceglatone, aldophosphamide glycoside, aminolevulinic acid, amsacrine, bestrabucil, bisantrene, edatraxate, defofamine, demecolcine, diaziquone, elformithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, tenuazonic acid, triaziquone, 2,2′,2″-trichlorotriethylamine, urethan, vindesine, dacarbazine, mannomustine, mitobronitol, mitolactol, pipobroman, gacytosine, arabinoside (Ara-C), cyclophosphamide, thiotepa, taxoids, e.g., paclitaxel, nab-paclitaxel and doxetaxel, chlorambucil, gemcitabine, 6-thioguanine, mercaptopurine, methotrexate, platinum and platinum coordination complexes such as cisplatin, oxaplatin and carboplatin, vinblastine, etoposide (VP-16), ifosfamide, mitomycin C, mitoxantrone, vincristine, vinorelbine, navelbine, novantrone, teniposide, daunomycin, aminopterin, xeloda, ibandronate, CPT11, topoisomerase inhibitors, difluoromethylornithine (DMFO), retinoic acid, esperamicins, capecitabine, taxanes such as paclitaxel, docetaxel, cabazitaxel, carminomycin, adriamycins such as 4′-epiadriamycin, 4-adriamycin-14-benzoate, adriamycin-14-octanoate, adriamycin-14-naphthaleneacetate, cholchicine and pharmaceutically acceptable salts, acids or derivatives of any of the above.

Chemotherapeutic agents may also include anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

The therapeutic agent may be an immunotherapeutic agent. Nonlimiting examples include programmed cell death protein 1 (PD-1) inhibitor, a cytotoxic T-lymphocyte-associated antigen 4 (CTLA-4) inhibitor, a programmed death-ligand 1 inhibitor (PD-L1), a lymphocyte activation gene 3 (LAG-3) inhibitor, a B- and T-lymphocyte attenuator (BTLA) inhibitor, an adenosine A2A (A2aR) inhibitor, or a B-7 family inhibitor. In some methods, the PD-1 inhibitor is an anti-PD-1 antibody. For example, and not to be limiting, the anti-PD-1 inhibitor can be selected from the group consisting of nivolumab, pembrolizumab and pidilizumab. In other methods, the anti-CTLA-4 inhibitor is an anti-CTLA-4 antibody. For example, and not to be limiting, the CTLA-4 inhibitor can be ipilimumab or tremelimumab.

The therapeutic agents described herein are administered in a number of ways depending on whether local or systemic treatment is desired. The compositions are administered via any of several routes of administration, including intraparenchymal injection, intravenously, intrathecally, intramuscularly, intracisternally, transdermally, or a combination thereof. Effective doses for any of the administration methods described herein can be extrapolated from dose-response curves derived from in vitro or animal model test systems.

Also provided herein is a method for determining progression of a disease (e.g., cancer) in a subject. The method includes administering a probe comprising the disclosed phosphorothioated DNA barcode sequence to the subject at a first timepoint. During the first timepoint, a first biological sample (e.g., blood or urine) may be obtained from the subject. DNA from the biological sample is extracted and a pair of forward and reverse primers, as described herein, that specifically recognize the DNA barcode sequence are used to amplify the DNA barcode to determine a first level of released DNA barcode. The DNA barcode may be amplified by various methods known in the art, for example amplification by PCR. The sequencing level of the DNA barcode may be determined by detecting the DNA barcode, for example by quantitative PCR or by counting the occurrences of the DNA barcode sequences (via its UMI sequence) for absolute molecular counting. Detection of the amplified DNA barcode sequences at the first time point provides a baseline for comparison.

At one or more subsequent timepoints (e.g., a second, a third, a fourth, etc. timepoint), the probe comprising the phosphorothioated DNA barcode sequence may be administered to the subject again and the process for extracting, amplifying, and detecting the amplified DNA barcode sequence repeated. The first DNA barcode sequence level can be compared to the second DNA barcode sequence level, and a determination of disease progression can be made. In one instance, the DNA barcode level may increase in the second biological sample as compared to the first biological sample, indicating the disease has progressed. In other instances, the DNA barcode level is comparable to (e.g., unchanged) or decreases in the second biological sample as compared to the first biological sample, indicating the disease has not progressed.

Provided herein is a method for determining the efficacy of a therapy for a disease (e.g., cancer) in a subject. The method is as described above for determining progression of a disease, but the subsequent time point or time points are selected to follow at least one treatment with a first therapy (e.g., a chemotherapeutic agent, an immunotherapeutic agent, radiation therapy, and/or a cellular therapy). Thus, the first or previous DNA barcode sequence level may be compared to the second or subsequent DNA barcode sequence level to determine efficacy of the treatment. Increased DNA barcode level from samples taken at subsequent timepoints as compared to the DNA barcode level in the biological sample at the first or prior timepoints indicates poor efficacy, whereas decreased DNA barcode level from samples taken at subsequent timepoints as compared to the DNA barcode level in the biological sample at the first or prior timepoints indicates efficacy. The results can then be used to determine whether modification of the treatment regime (e.g., a change in the agent or the dosing regimen) is desired.

Also provided herein is a kit comprising (a) a probe disclosed herein, (b) a forward primer comprising a nucleic acid sequence that comprises about 18 nucleotides that hybridize to about 18 nucleotides at the 3′ end of the DNA barcode, and (c) a reverse primer comprising a nucleic acid sequence that comprises about 18 nucleotides that hybridize to about 18 nucleotides at the 5′ end of the DNA barcode. The probe in the kit may be attached to or contained within a nanoparticle as described herein.

Optionally, the kit includes multiple sets of probes with each set having phosphorothioated DNA barcodes of different lengths. For example, the kit may include probes with DNA barcodes of about 36 nucleotides to about 72 nucleotides in length, about 36 nucleotides to about 62 nucleotides in length, about 36 nucleotides to about 46 nucleotides in length, or any combination thereof.

The kits can include probes with barcodes, wherein the barcodes are about 36-72nucleotides in length, including for example, 72, 62, 46, or 36 nucleotides in length in addition to one or any combination of the following: (i) 5′ and 3′ priming sequences at the 5′ end and 3′ end, respectively, of the DNA barcode sequence, wherein each priming sequence comprises about 18 nucleotides, (ii) a UMI sequence of about 10 nucleotides, (iii) a sequence used for NGS sequencing and/or library management (e.g., Sseq+Nseq) comprising about 16 nucleotides, and (iv) a spacer sequence of about 10 nucleotides that represents the nucleic acids outside of the priming regions. The about 72, 62, 46, and/or 36 nucleotide long DNA barcodes comprise at least one phosphorothioate-modifications (e.g., in the 3′ and/or 5′ priming sequences or lacking in the priming sequences), phosphorothioate-modifications at every base or at intervals (e.g., every second or fourth base). See Table 1 for examples.

Although Table 1 shows various lengths of the same base sequence (e.g., AGCGTCAGATGTGTATAATCGGAAGAGCGGTTCAGC (SEQ ID NO: 4), it should be understood by one skilled in the art, that any DNA barcode sequence comprising about 36nucleotides to about 72 nucleotides in length, about 36 nucleotides to about 62 nucleotides in length, or about 36 nucleotides to about 46 nucleotides in length may be contemplated that also comprise either (i) at least one phosphorothioate-modifications in the 3′ and 5′ priming sequences, (ii) phosphorothioate-modifications at every second based, (iii) phosphorothioate-modifications at every fourth base, (iv) phosphorothioate-modifications at every base, or (v) the 3′ and 5′ priming sequences are not phosphorothioated modified. The DNA barcode sequence may comprise any DNA sequence not found in nature and may be generated randomly.

Accordingly, the sequences of the forward primer and the reverse primer that hybridize to the 3′ end and 5′ end, respectively, of the DNA barcode will be dependent on the sequence of the DNA barcode.

### Additional Definitions

As used herein, the articles ‘a’ and ‘an’ are used herein to refer to one or to more than one (i.e. at least one) of the grammatical object of the article. By way of example, an element means at least one element and can include more than one element.

The term about is used to provide flexibility to a numerical range endpoint by providing that a given value may be slightly above or slightly below the endpoint without affecting the desired result.

The use herein of the terms including, comprising, or having, and variations thereof, is meant to encompass the elements listed thereafter and equivalents thereof as well as additional elements. Embodiments recited as including, comprising, or having certain elements are also contemplated as consisting essentially of and consisting of those certain elements. As used herein, and/or, refers to and encompasses any and all possible combinations of one or more of the associated listed items, as well as the lack of combinations were interpreted in the alternative (or).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example 1

### Reagents and Materials

DNA barcodes were created and shipped lyophilized. They were rehydrated in TE buffer (Tris-EDTA) pH 8.0 and then diluted in Phosphate Buffered Saline (PBS) to the desired concentration for injection into mouse models (e.g., 100 microliter injection volume, containing 1 microgram of DNA). Mouse urine was later collected on paper bedding and extracted using water washes (e.g., two washes using 3mL water per wash) and centrifuged. DNA was isolated from the wash water using complement-capture magnetic beads. The target DNA was later released from the capture beads using sodium hydroxide, which was then neutralized with hydrochloric acid, and then added to a PCR reaction for quantitation of target DNA.

### Example 2

### RTqPCR Experimental Set Up

Quantitative PCR reactions were run in 96-well plates using a Bio-RAD CFX real time qPCR machine, according to the manufacturer's protocol. Each well contained 5 μL SYBR master mix, 2 μL water, 1 μL neutralized extracted DNA (or diluted template), and 2 μL diluted primers.

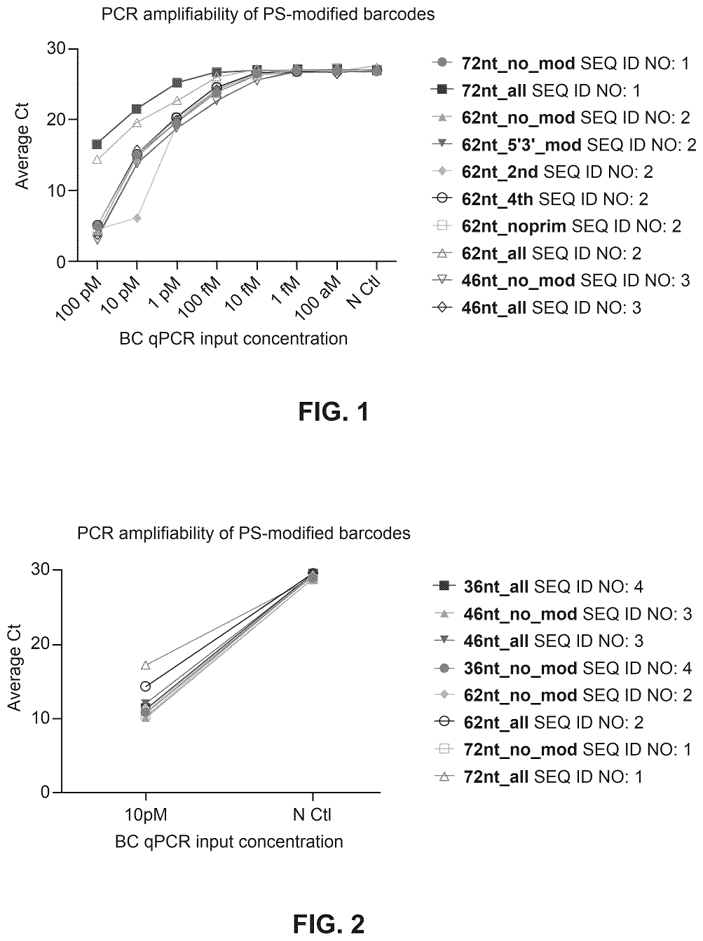

The above-described method was used to generate the qPCR data illustrated in FIG. 1 and FIG. 2 for SEQ ID NOs: 1-4. As shown in FIG. 1, both the 72 nucleotide and 62 nucleotide fully PS-modified DNA (all) had significantly higher Ct values compared to their corresponding non-modified and partially modified barcodes. Unexpectedly, the 46 nucleotide fully modified barcode (all) had Ct values more similar to the non-modified barcodes compared to the fully modified barcodes, supporting that the length of the barcode was critical to PCR detection of the probes. Table 2 provides the raw RTqPCR data values for FIG. 1 while Table 3 provides the raw RTqPCR data values for FIG. 2.

### Example 3

### Fragment Analysis

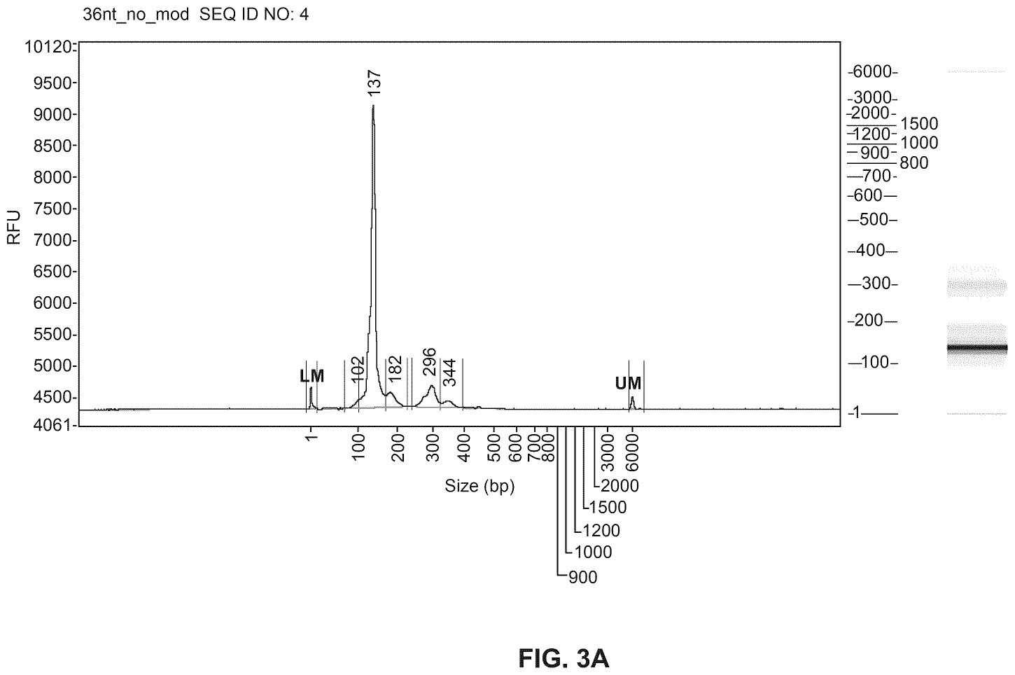

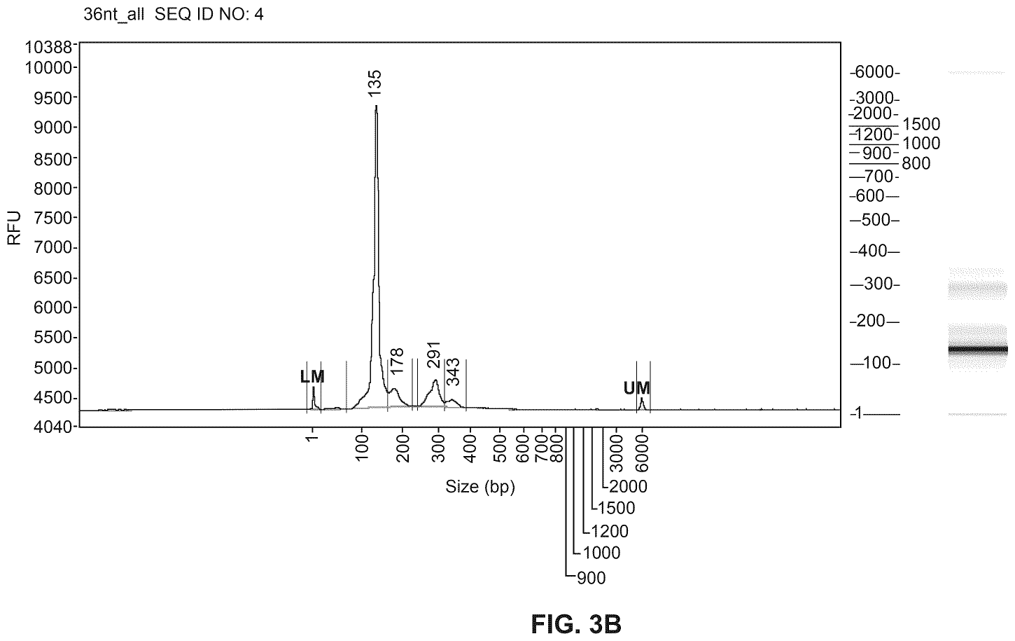

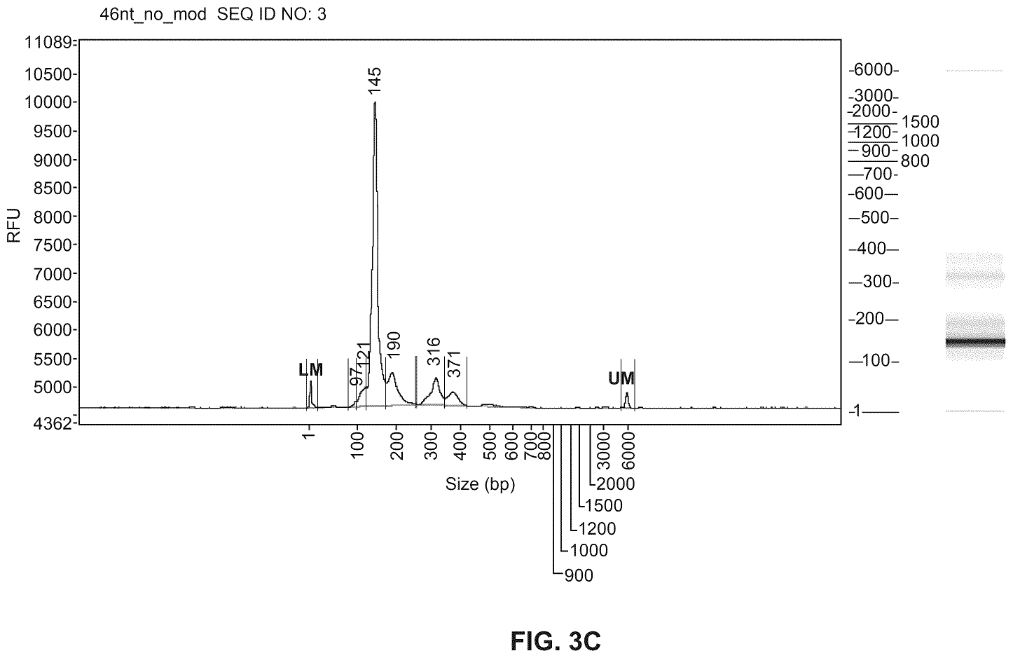

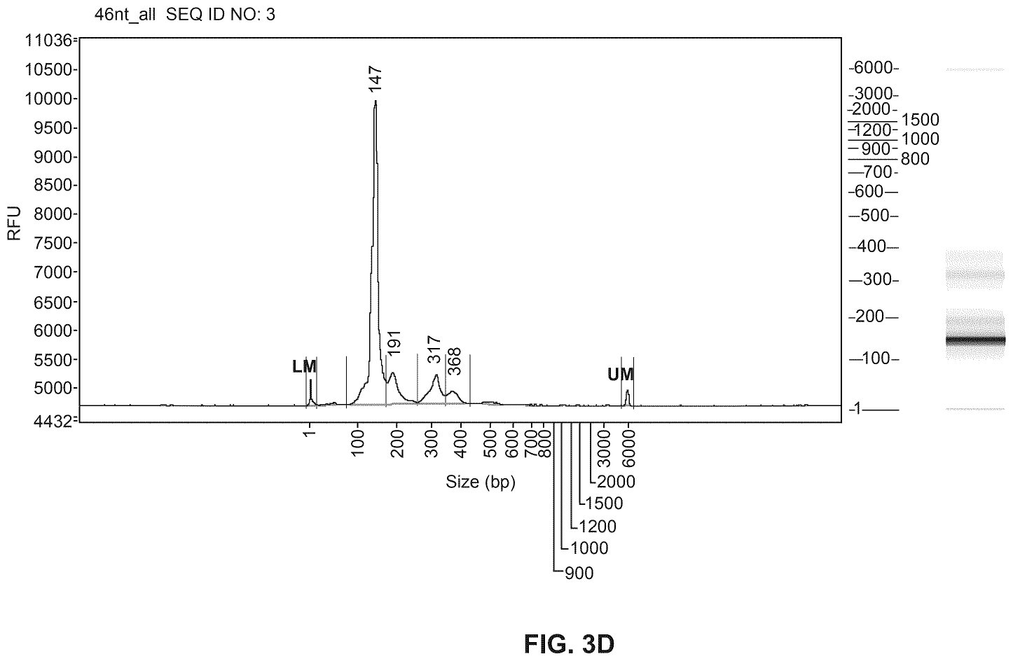

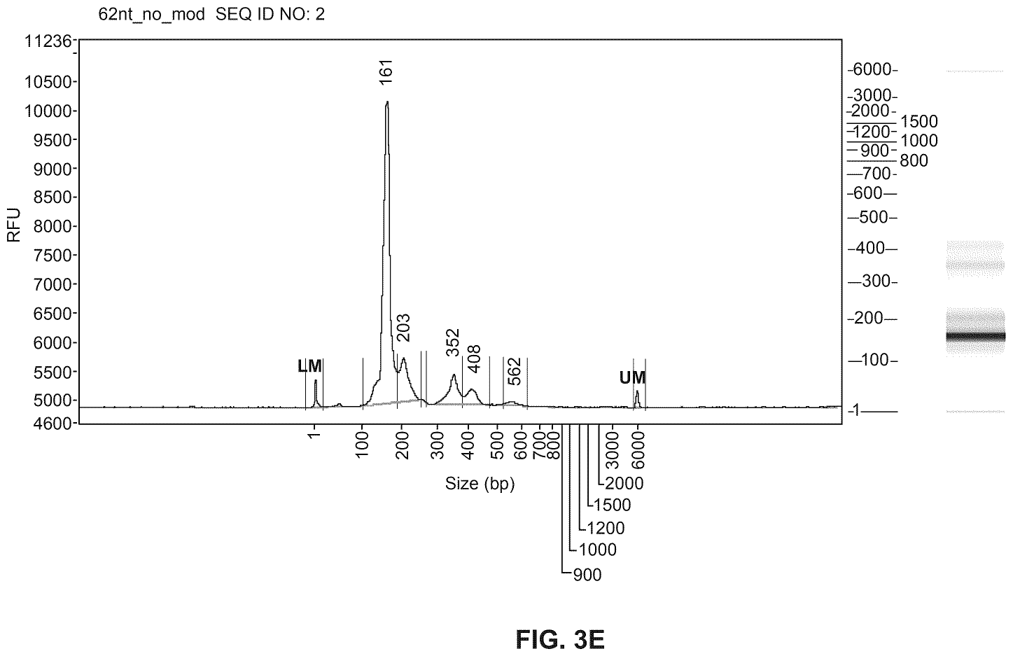

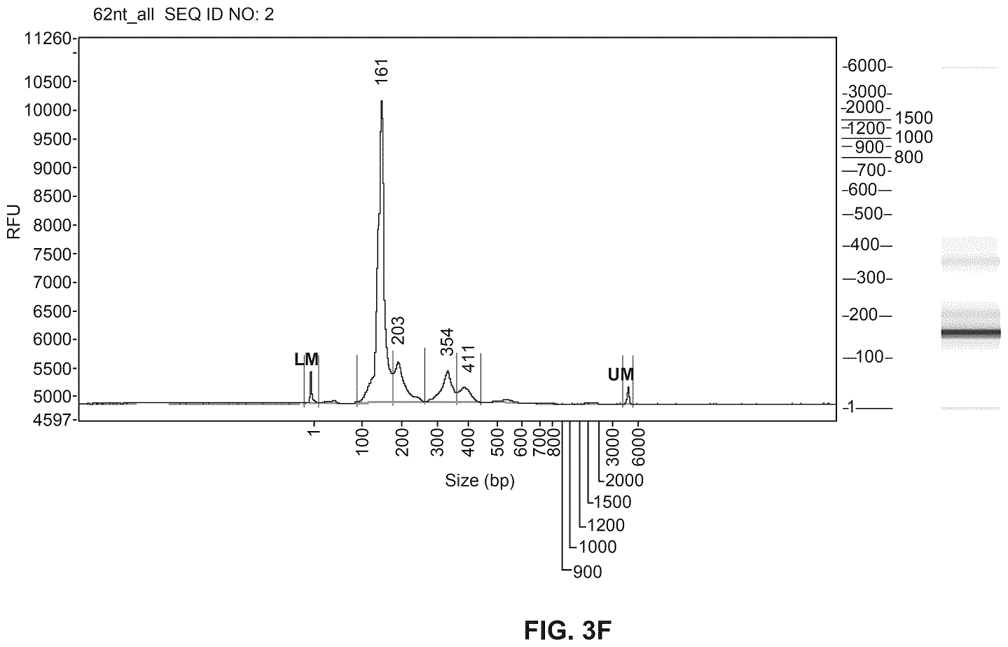

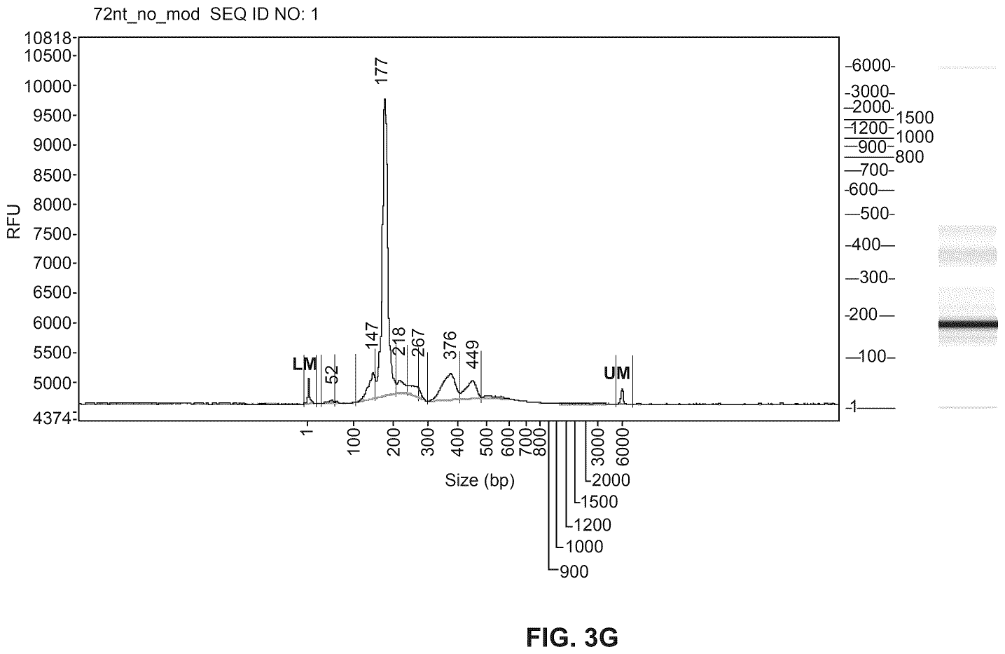

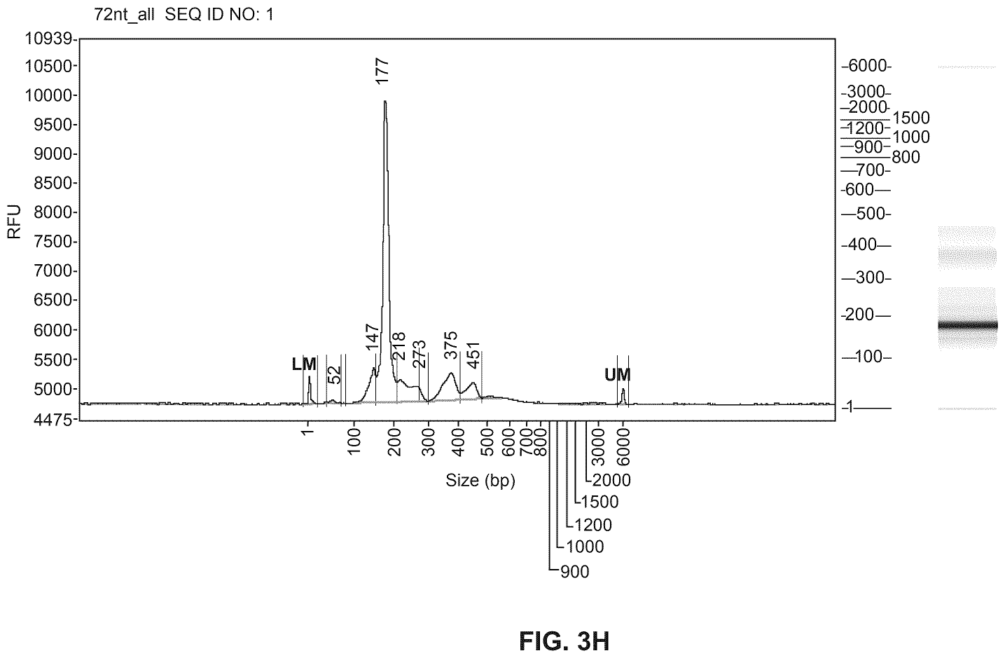

Fragment analysis was performed using a qSep DNA fragment analyzer to determine the size distribution of DNA produced by the qPCR process to confirm that amplification occurred on the intended template, and not primer dimers or other unwanted amplicons. FIG. 3A-3H display the fragment analysis for various modified versions of SEQ ID NOs: 1-4. Each figure demonstrates a prominent peak corresponding to the appropriate amplicon length.

What is claimed:

1. A method for detecting the presence of a disease in a subject comprising:
(a) administering to the subject a probe comprising a DNA barcode, wherein the DNA barcode comprises about 36 nucleotides to about 72 nucleotides, wherein the DNA barcode comprises at least one phosphorothioated modification, and wherein the DNA barcode is released when the disease is present in the subject;
(b) obtaining a biological sample containing the released DNA barcode from the subject; and
(c) amplifying and detecting the released DNA barcode in the biological sample, wherein detection of the amplified DNA barcode sequence in the biological sample indicates the presence of the disease in the subject.

2. The method of claim 1, wherein the DNA barcode comprises a priming sequence at the 5′ end of the DNA barcode sequence, a priming sequence at the 3′ end of the DNA barcode sequence, and a unique molecular identifier sequence.

3. The method of claim 2, wherein the DNA barcode comprises:
a priming sequence comprising about 18 nucleotides at the 5′ end of the DNA barcode sequence and a priming sequence comprising about 18 nucleotides at the 3′ end of the DNA barcode sequence; and
a unique molecule identifier.

4. The method of claim 1, wherein the DNA barcode is either single stranded or double stranded DNA.

5. The method of claim 1, wherein the DNA barcode comprises at least one phosphorothioated modification at the 3′ end of the DNA barcode and at least one phosphorothioated modification at the 5′ end of the DNA barcode.

6. The method of claim 1, wherein the DNA barcode comprises a phosphorothioated modification at every second nucleotide.

7. The method of claim 1, wherein the DNA barcode comprises a phosphorothioated modification at every fourth nucleotide.

8. The method of claim 1, wherein the DNA barcode comprises a phosphorothioated modification at every nucleotide.

9. The method of claim 1, wherein the priming sequences are not phosphorothioated modified.

10. The method of claim 1, wherein the unique molecular identifier sequence is between about five to about ten nucleotides in length.

11. The method of claim 1, wherein the disease is cancer.

12. The method of claim 1, wherein the biological sample is a urine sample.

13. The method of claim 1, wherein amplification is performed by polymerase chain reaction (PCR).

14. The method of claim 1, wherein the DNA barcode sequence is amplified by a forward primer comprising a nucleic acid sequence that comprises about 18 nucleotides that hybridize to the priming sequence at the 3′ end of the DNA barcode; and a reverse primer comprising a nucleic acid sequence that comprises about 18 nucleotides that hybridize to the priming sequence at the 5′ end of the DNA barcode.

15. The method of claim 1, wherein the probe further comprises an enzymatic cleavage site operably linked to the DNA barcode.

16. The method of claim 15, wherein the enzymatic cleavage site is recognized by an enzyme associated with the disease in the subject.

17. The method of claim 1, wherein the probe is attached to or contained within a nanoparticle.

18. A method of treating a disease in a subject comprising:
(a) performing the method of claim 1 to detect the presence of the disease in the subject; and
(b) treating the disease in the subject by administering a therapeutic agent to the subject.

19. The method of claim 18, wherein the disease is cancer.

20. A method for determining the efficacy of a therapy for a disease in a subject comprising:
(a) performing steps (a)-(c) of claim 1 at a first timepoint to determine a first level of amplified DNA barcode in a first sample;
(b) administering a therapy to the subject after the first timepoint;
(c) repeating steps (a)-(c) of claim 1 after at a second timepoint after administration of the therapy to determine a second level of amplified DNA barcode in a second sample;
(d) comparing the first level of amplified DNA barcode level to the second level of amplified DNA barcode; and
(e) administering the same therapy or a different therapy if the second level of amplified DNA barcode is higher, lower, or unchanged as compared to the first level of amplified DNA barcode.


SEQUENCE LISTING (4 sequences)

SEQ ID NO: 1 - DNA, 72 bp, synthetic construct
  source 1..72: mol_type = other DNA
agcgtcagatgtgtataagagacagnnnnnnnnnnctctctattaaggcgaagatcggaagagcggttcagc

SEQ ID NO: 2 - DNA, 62 bp, synthetic construct
  source 1..62: mol_type = other DNA
agcgtcagatgtgtataagagacagctctctattaaggcgaagatcggaagagcggttcagc

SEQ ID NO: 3 - DNA, 46 bp, synthetic construct
  source 1..46: mol_type = other DNA
agcgtcagatgtgtataagagacagagatcggaagagcggttcagc

SEQ ID NO: 4 - DNA, 36 bp, synthetic construct
  source 1..36: mol_type = other DNA
agcgtcagatgtgtataatcggaagagcggttcagc